(19) United States
(12) Patent Application Publication
(10) Pub. No.: US 2017/0333305 A1
(43) Pub. Date: Nov. 23, 2017

(54) TOPICAL SKIN PRODUCT HAVING RETENTION PROPERTY

(71) Applicant: Microban Products Company, Huntersville, NC (US)

(72) Inventors: Ivan W. Ong, Charlotte, NC (US); Tian Lan, Huntersville, NC (US); Gina Parise Sloan, Statesville, NC (US); Karen Terry Welch, Kannapolis, NC (US)

(21) Appl. No.: 15/600,908

(22) Filed: May 22, 2017

Related U.S. Application Data
(60) Provisional application No. 62/340,300, filed on May 23, 2016.

(51) Int. Cl. A61K 8/18; A61K 8/02; A61K 8/35; A61Q 19/00

(52) U.S. Cl. CPC A61K 8/18; A61Q 19/008; A61Q 19/007; A61K 8/02; A61K 8/35; A61Q 19/005

(57) ABSTRACT

A topical skin product having a retention property is provided. The topical skin product comprises a retention ingredient. A suitable retention ingredient is an oxazoline homopolymer or an extended or a modified polymer based on an oxazoline homopolymer. The topical skin product may be a hand sanitizer having a gel-based hand sanitization formulation.

### CROSS-REFERENCE TO RELATED APPLICATIONS

The present application claims priority from U.S. provisional patent application 62/340,300, filed on May 23, 2016, incorporated by reference herein in its entirety.

### FIELD OF THE INVENTION

The present invention generally relates to a topical skin product, more particularly to a topical skin product having a retention property.

### BACKGROUND OF THE INVENTION

There are a vast number of topical skin products on the market. Among the topical skin products on the market are hand sanitizers. Most hand sanitizers contain alcohol as the primary ingredient for killing germs on hands.

Current alcohol-based and water-based hand sanitizers suffer from a number of issues such as hand dry out, antimicrobials are not activated or effective due to dry hands, the sanitization effect is not long lived, the product is easily removed from the hands, among other issues.

Typical hand sanitizers having predominantly alcohol and water-based formulations are not able to be retained on the skin for long term once dried. Alcohol provides a quick kill but evaporates quickly, typically in a few seconds.

There are other current challenges with alcohol and water-based hand sanitizers. Such hand sanitizers are utilized everyday to remove bacteria from hands. However, these sanitizers only have temporary activity. To allow for prolonged activity some manufacturers have been adding in a quaternary ammonium compound, specifically benzalkonium chloride. While this passes standard methods, it is unlikely to provide a long lasting benefit under real world conditions.

Thus, there is a need for a hand sanitizer or other topical skin product that is formulated to have beneficial properties that can be experienced longer than what current formulations offer and overcomes these challenges.

### SUMMARY OF THE INVENTION

The present invention relates to a topical skin product having a retention property. The topical skin product comprises a retention ingredient. A suitable retention ingredient is an oxazoline homopolymer or an extended or a modified polymer based on an oxazoline homopolymer.

In an embodiment of the invention, the topical skin product is a hand sanitizer having a gel-based hand sanitization formulation.

In an embodiment of the invention, a polymeric binder is present in the topical skin product. The polymeric binder is non-immunogenic and immobilizes a quaternary ammonium onto the skin, providing longer control of bacteria on the skin.

Further areas of applicability of the present invention will become apparent from the detailed description provided hereinafter. It should be understood that the detailed description and specific examples, while indicating the preferred embodiments of the invention, are intended for purposes of illustration only and are not intended to limit the scope of the invention.

### DESCRIPTION OF THE PREFERRED EMBODIMENTS

The following description of the embodiments of the present invention is merely exemplary in nature and is in no way intended to limit the invention, its application, or uses. The present invention has broad potential application and utility, which is contemplated to be adaptable across a wide range of industries. The following description is provided herein solely by way of example for purposes of providing an enabling disclosure of the invention, but does not limit the scope or substance of the invention.

As used herein, the terms “microbe” or “microbial” should be interpreted to refer to any of the microscopic organisms studied by microbiologists or found in the use environment of a treated article. Such organisms include, but are not limited to, bacteria and fungi as well as other single-celled organisms such as mold, mildew and algae. Viral particles and other infectious agents are also included in the term microbe.

“Antimicrobial” further should be understood to encompass both microbicidal and microbistatic properties. That is, the term comprehends microbe killing, leading to a reduction in number of microbes, as well as a retarding effect of microbial growth, wherein numbers may remain more or less constant (but nonetheless allowing for slight increase/decrease).

For ease of discussion, this description uses the term antimicrobial to denote a broad spectrum activity (e.g. against bacteria and fungi). When speaking of efficacy against a particular microorganism or taxonomic rank, the more focused term will be used (e.g. antifungal to denote efficacy against fungal growth in particular).

Using the above example, it should be understood that efficacy against fungi does not in any way preclude the possibility that the same antimicrobial composition may demonstrate efficacy against another class of microbes.

For example, discussion of the strong bacterial efficacy demonstrated by a disclosed embodiment should not be read to exclude that embodiment from also demonstrating antifungal activity. This method of presentation should not be interpreted as limiting the scope of the invention in any way.

Further, the term “or” as used in this disclosure and the appended claims is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form. Throughout the specification and claims, the following terms take at least the meanings explicitly associated herein, unless the context dictates otherwise. The meanings identified below do not necessarily limit the terms, but merely provided illustrative examples for the terms. The meaning of “a,” “an,” and “the” may include plural references, and the meaning of “in” may include “in” and “on.” The phrase “in one embodiment,” as used herein does not necessarily refer to the same embodiment, although it may.

In an embodiment of the present invention, a topical skin product having a retention property is provided. Examples of topical skin products include, but are not limited to, hand sanitizers, body treatments, sun tan products, hair products, body moisturizers, cosmetic face products, treatment ointments (such as for acne care, wound care, insect bites, rashes, among others).

The topical skin product can be in a variety of forms including, but not limited to, gel, liquid, cream, lotion, aerosol, and roll-on.

The term “retention property”, as used herein, generally refers to a capability to provide a prolonged presence of ingredients in contact with the skin. In an embodiment of the invention, a hand sanitizer formulation comprises a retention ingredient. The use of a retention ingredient creates a moisture rich barrier film that potentiates antimicrobial activity and promotes skin welfare by preventing dry off. The retention ingredient is conducive to maintaining moisturized skin, for example. The use of a retention ingredient aids in retention of antimicrobials, adjuvants such as moisturizers, Vitamin E, UV absorbers, and therapeutic agents that promote skin health and healing. It is preferred that the retention ingredient is non-permanent and easily removed by washing. The retention ingredient is selected to be low toxicity and skin friendly.

It is contemplated that the topical skin product of the invention may comprise other ingredients such as moisturizers and/or other skin desirable ingredients.

In an embodiment of the invention, the retention ingredient itself is hydrophilic, thereby retaining a certain amount of moisture and aiding in moisture retention.

In an embodiment of the invention, the retention ingredient is an oxazoline homopolymer. As another feature of the invention, the oxazoline homopolymer has the following structure:

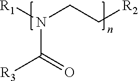

wherein

R₁ and R₂ are end groups determined by the polymerization techniques used to synthesize oxazoline homopolymer. R₁ and R₂ are independently selected and include, but are not limited to, hydrogen, alkyl, alkenyl, alkoxy, alkylamino, alkynyl, allyl, amino, anilino, aryl, benzyl, carboxyl, carboxyalkyl, carboxyalkenyl, cyano, glycosyl, halo, hydroxyl, oxazolinium mesylate, oxazolinium tosylate, oxazolinium triflate, silyl oxazolinium, phenolic, polyalkoxy, quaternary ammonium, thiol, or thioether groups. Alternatively, R₂ could include a macrocyclic structure formed during synthesis as a consequence of intramolecular attack.

For example, R₁ is a methyl group and R₂ is oxazolinium tosylate if methyl tosylate is used as the initiator in the cationic initiated polymerization of oxazoline.

R₃ is an end group determined by the type of oxazoline used in the preparation of the retention ingredient of this invention. R₃ includes, but is not limited to, hydrogen, alkyl, alkenyl, alkoxy, aryl, benzyl, hydroxyalkyl, or perfluoroalkyl. For example, R₃ is an ethyl group if ethyloxazoline is the monomer used to prepare the retention ingredient for the present invention.

n is the degree of oxazoline polymerization in the homopolymer. n is in a range of 1 to 1,000,000. Preferably, n is in a range of 500 to 250,000; most preferably, n is in a range of 2500 to 100,000.

Similar to oxazoline homopolymer, extended or modified polymers with some variations based on the oxazoline homopolymer are also suitable for the present invention. The techniques and options for performing chemical or molecular structure variations or modifications to oxazoline should be familiar to those skilled in the art. A class of extended or modified polymers based on oxazoline homopolymer can be represented with the following molecular structure:

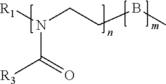

wherein

B is additional monomer repeating unit linked to oxazoline in a coploymer. The types of arrangement of the repeating units between B and oxazoline in the copolymer can include, but are not limited to, block, alternating, periodic, or combinations thereof. There is no limitation as to the types of B that can be used to copolymerize with or modify the oxazoline of the present invention.

n is the degree of polymerization for an oxazoline repeating unit; n in the copolymer is in a range of 1 to 1,000,000 and the degree of polymerization for B repeating unit in the copolymer m is in a range of 0 to 500,000 at the same time. Preferably, n is in a range of 500 to 250,000 and m is in a range of 20 to 10,000; and most preferably, n is in a range of 2500 to 100,000 and m is in a range of 50 to 5,000. In addition to linking B to ethyloxazoline through copolymerization, B could also be linked to oxazoline as an end group in a cationic polymerization by using B as a cationic initiator if B itself is already a quaternary ammonium compound.

Not intended to be all inclusive, B can be, for example, ethyleneimine with the following molecular structure:

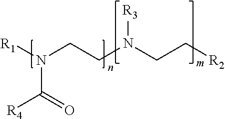

wherein

R₁ and R₂ end groups have the same definition as those outlined for oxazoline homopolymer.

R₃ includes, but is not limited to, hydrogen, alkyl, alkenyl, alkoxy, aryl, benzyl, hydroxyalkyl, or perfluoroalkyl.

R₄ includes, but is not limited to, hydrogen, alkyl, alkenyl, alkoxy, aryl, benzyl, hydroxyalkyl, or perfluoroalkyl.

m is in a range of 0 to 500,000; preferably, in a range of 20 to 10,000; and most preferably, in a range of 50 to 5,000.

n is in a range of 1 to 1,000,000; preferably, 500 to 250,000; most preferably, in a range of 2500 to 100,000.

The synthesis of oxazoline and ethyleneimine copolymer can be phased into two steps, for example. In a first step, a cationic ring opening polymerization technique can be used to make polyoxazoline homopolymer. In a second step, the polyoxazoline made in the first step can be hydrolyzed to convert part of polyoxazoline repeating units into polyethyleneimine. Alternatively, oxazoline-ethylenimine copolymer can be made with the appropriate respective monomers, an oxazoline and an aziridine. The result would be a cationic polymer having the above structure.

The degree of polymerization for oxazoline repeating unit n in the copolymer is in a range of 1 to 1,000,000 and the degree of polymerization for ethyleneimine repeating unit in the copolymer m is in a range of 0 to 500,000 at the same time. Preferably, n is in a range of 500 to 250,000 and m is in a range of 20 to 10,000, and most preferably n is in a range of 2500 to 100,000 and m is in a range of 50 to 5,000.

Alternatively, the nitrogen in the ethyleneimine repeating unit could be further quarternized to generate the following cationic copolymer:

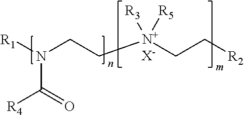

Any quaternization technique that is familiar to those skilled in the art could be used to quaternize the polymer of this example. R_{1,} R_{2,} R₃ and R₄ have the same meaning as those designated in the above oxazoline-ethyleneimine copolymer. R₅ includes, but is not limited to, a hydrogen, methyl, ethyl, propyl, or other types of alkyl group. The corresponding anion X⁻ is a halogen, sulfonate, sulfate, phosphonate, phosphate, carbonate/bicarbonate, hydroxy, or carboxylate.

The ranges for n and m are also the same as those described in oxazoline-ethyleneimine copolymer.

Another example of B that can be used for the present invention is polydiallyldimethylammonium chloride. Polyethyloxazoline modified with polydiallyldimethylammonium chloride has the following structure:

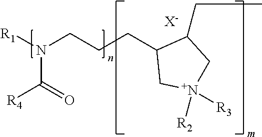

wherein

R₁ and R₄ have the same meaning as described in previous example for quarternized oxazoline-ethyleneimine copolymer.

R₂ and R₃, independently, include, but are not limited to, short chain alkyl groups such as C₁ to C_{6.} The corresponding anion X⁻ is a halogen, sulfonate, sulfate, phosphonate, phosphate, carbonate/bicarbonate, hydroxy, or carboxylate.

n and m are defined and numbered the same as in previous examples.

B could be other olefins including, but not limited to, diallyldimethylammonium chloride, styrene, methoxystyrene, and methoxyethene. Ethyloxazoline can also be copolymerized with heterocyclic monomers such as oxirane, thietane, 1,3-dioxepane, oxetan-2-one, and tetrahydrofuran to enhance the performance of the polymer for the present invention. The binder used in this invention could also employ pendant oxazoline groups on a polymer backbone, such as an acrylic or styrene based polymer, or a copolymer containing acrylic or styrene.B could be other olefins including, but not limited to, diallyldimethylammonium chloride, styrene, methoxystyrene, and methoxyethene. Ethyloxazoline can also be copolymerized with heterocyclic monomers such as oxirane, thietane, 1,3-dioxepane, oxetan-2-one, and tetrahydrofuran to enhance the performance of the polymer for the present invention. The binder used in this invention could also employ pendant oxazoline groups on a polymer backbone, such as an acrylic or styrene based polymer, or a copolymer containing acrylic or styrene.

Examples of commercially available polyethyloxazolines include, but are not limited to, Aquazol 500 from Polymer Chemistry Innovations, Inc.

In an embodiment of the invention, a hand sanitizer is provided. In a preferred embodiment, the hand sanitizer is a hand gel sanitization formulation. The hand sanitizer generally comprises a retention ingredient. Use of a retention ingredient in hand sanitizer formulations creates a retention coating for antimicrobials and other desirable adjuvants such as moisturizing components so as to create a more long lasting protection.

In a preferred embodiment of the invention, the retention ingredient is present in the hand sanitizer formulation in an amount in a range of greater than 0% to 10%, based on the weight of the hand sanitizer formulation.

The hand sanitizer may further comprise an antimicrobial. Examples of antimicrobials include, but are not limited to, triclosan, chlorhexadiene, and quaternary ammonium compounds such as benzalkonium chloride and benzethonium chloride, triclocarbon, povidone-iodine, silver (nano silver, silver salts such as silver chloride, silver in zeolites, silver in dissolvable glass particles), antimicrobials derived from plants (tea tree oil, etc.).

In an embodiment of the invention, the quaternary ammonium compound has the following molecular structure:

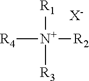

wherein

R_{1,} R_{2,} R_{3,} and R₄ are independently selected and include, but are not limited to, alkyl, alkoxy, or aryl, either with or without heteroatoms, or saturated or non-saturated. Some or all of the functional groups may be the same.

The corresponding anion X⁻ includes, but is not limited to, a halogen, sulfonate, sulfate, phosphonate, phosphate, carbonate/bicarbonate, hydroxy, or carboxylate.

QACs include, but are not limited to, n-alkyl dimethyl benzyl ammonium chloride, di-n-octyl dimethyl ammonium chloride, dodecyl dimethyl ammonium chloride, n-alkyl dimethyl benzyl ammonium saccharinate, and 3-(trimethoxysilyl) propyldimethyloctadecyl ammonium chloride.

Combinations of monomeric QACs may be used for the invention. A specific example of QAC combination is N-alkyl dimethyl benzyl ammonium chloride (40%); N-octyl decyl dimethyl ammonium chloride (30%); di-n-decyl dimethyl ammonium chloride (15%); and di-n-dioctyl dimethyl ammonium chloride (15%). The percentage is the weight percentage of individual QAC based on the total weight of blended QACs composition.

Polymeric version of the QACs with the following structures may also be used for the invention.

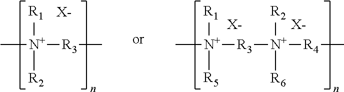

wherein

R₁, R₂, R₅, and R_{6,} independently, include, but are not limited to, hydrogen, methyl, ethyl, propyl or other longer carbon alkyl groups.

R₃ and R₄ are independently selected and include, but are not limited to, methylene, ethylene, propylene or other longer alkylene linking groups.

n is the degree of polymerization; n is an integer in a range of from 2 to 10,000.

Examples of cationic polymers with the above structure, include but are not limited to, polyamines derived from dimethylamine and epichlorohydrin such as Superfloc C-572 commercially available from Kemira Chemicals.

Still another polymeric QAC that may be suitable for the invention is poly diallyldimethylammonium chloride or polyDADMAC.

Yet another class of QACs that may be useful for the present invention are those chemical compounds with biguanide moiety in the molecule. Examples of this class of cationic antimicrobials include, but are not limited to, PHMB and chlorhexidine.

Examples of commercially available quaternary ammonium compounds include, but are not limited to, Bardac 205M and 208M from Lonza, and BTC885 from Stepan Company.

The hand sanitizer may comprise other additives including, but not limited to, vitamins, SPF sun block, and moisturizers. The polymer binder may be present by itself in combination with the carrier and the quaternary ammonium compound, or may be used in conjunction with any cationic moisturizing ingredient in combination with the carrier and the quaternary ammonium compound to improve skin barrier function. For example, the polymer binder may serve as a host for moisture beads, UV protectants, antimicrobials, or other additives to promote skin health including, but not limited to, vitamins, aloe, polysaccharides or other naturally derived ingredients.

The hand sanitizer may comprise a carrier. Carriers include, but are not limited to, water, alcohol, and a combination thereof. The hand sanitizer formulation can be with or without an alcohol. Alcohol can be present in a range of 0% up to 70% by weight of the formulation. Alcohol can be in a range of 60% to 70% by weight of the formulation. The alcohol may also be present in relatively low concentrations because it is not being relied upon for making the full load of the kill for purposes of sanitization.

Applications (i.e. end uses) for the topical skin product of the invention include, but are not limited to, hand sanitizers, hair gels, body moisturizers, sun tan lotions, cosmetic face masks, topical skin treatments in ointments (such as acne) and wound care (such as antibiotic creams) to hold on to desired chemicals by forming a film, insect repellants, and insect bite cream.

The topical skin product of the present invention forms a breathable barrier film that allows passage of moisture yet is hydrophilic enough to prevent dryness. The polymeric binder added to a system that is non-immunogenic provides a way to immobilize the quaternary ammonium onto the skin, providing longer control of bacteria on the skin.

### EXAMPLES

Comparative experiments were conducted to test the retention property of the following products used as topical hand sanitizers: (1) commercially available 70 weight % alcohol gel (Purel); (2) 70 weight % alcohol gel (Purel) with 0.1 weight % quaternary ammonium (Stepan BTC 885); and (3) 70 weight % alcohol gel (Purel) with 0.1 weight % quaternary ammonium (Stepan BTC 885) with 1 weight % polymer binder (Aquazol 500).

Three volunteers were selected. Each volunteer utilized one of the products listed above. All volunteers were allowed to continue with normal daily activities while conducting the experiment. Samples were taken by volunteers placing their hand onto Tryptic Soy Agar (TSA) with 0.01 weight % triphenyl tetrazolium chloride (TTC) as a viable dye indicator. TTC is typically clear but in the presence of viable microorganisms it is metabolized into a red pigment. The presence of a red pigment indicated the presence of viable organisms. Each volunteer was sampled at four different times:(1) immediately before product application; (2) five minutes after product application; (3) two hours after product application; and (4) 5.5 hours after product application.

The alcohol gel product alone controlled removed viable organisms when used per instructions. However, the bacteria rebounded and surpassed the original contamination levels by 5.5 hours after product application. The addition of quaternary ammonium to the alcohol gel did not improve efficacy as per label claims.

The addition of quaternary ammonium and the polymer binder had a long lasting effect on viable organisms over the testing timeframe. The combination product was able to provide a sustained reduction even 5.5 hours after the product was applied. There was a synergistic effect with (3) having the combination with the polymer binder.

It will therefore be readily understood by those persons skilled in the art that the present invention is susceptible of broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements.

What is claimed is:

1. A topical skin product comprising:
a topical skin composition; and
a retention ingredient, wherein the retention ingredient is an oxazoline homopolymer or an extended or a modified polymer based on an oxazoline homopolymer.

2. The topical skin product according to claim 1, wherein the retention ingredient is present in the topical skin product in an amount in a range of greater than 0% to 10%, based on the weight of the topical skin product.

3. The topical skin product according to claim 1, wherein the oxazoline homopolymer has a structure of: 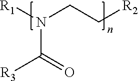
wherein
R₁ is a hydrogen, alkyl, alkenyl, alkoxy, alkylamino, alkynyl, allyl, amino, anilino, aryl, benzyl, carboxyl, carboxyalkyl, carboxyalkenyl, cyano, glycosyl, halo, hydroxyl, oxazolinium mesylate, oxazolinium tosylate, oxazolinium triflate, silyl oxazolinium, phenolic, polyalkoxy, quaternary ammonium, thiol, or thioether group;
R₂ is a hydrogen, alkyl, alkenyl, alkoxy, alkylamino, alkynyl, allyl, amino, anilino, aryl, benzyl, carboxyl, carboxyalkyl, carboxyalkenyl, cyano, glycosyl, halo, hydroxyl, oxazolinium mesylate, oxazolinium tosylate, oxazolinium triflate, silyl oxazolinium, phenolic, polyalkoxy, quaternary ammonium, thiol, or thioether group or a macrocyclic structure;
R₃ is a hydrogen, alkyl, alkenyl, alkoxy, aryl, benzyl, hydroxyalkyl, or perfluoroalkyl group; and
n is in a range of 1 to 1,000,000.

4. The topical skin product according to claim 1, wherein the topical skin composition comprises a carrier and a quaternary ammonium compound.

5. The topical skin product according to claim 4, wherein the carrier is selected from the group consisting of water, alcohol, and a combination thereof.

6. The topical skin product according to claim 4, wherein the quaternary ammonium compound has a structure of: 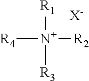
wherein
R₁ is alkyl, alkoxy, or aryl, either with or without heteroatoms, or saturated or non-saturated;
R₂ is alkyl, alkoxy, or aryl, either with or without heteroatoms, or saturated or non-saturated;
R₃ is alkyl, alkoxy, or aryl, either with or without heteroatoms, or saturated or non-saturated;
R₄ is alkyl, alkoxy, or aryl, either with or without heteroatoms, or saturated or non-saturated;
X⁻, an anion, is halogen, sulfonate, sulfate, phosphonate, phosphate, carbonate, bicarbonate, hydroxy, or carboxylate.

7. The topical skin product according to claim 4, wherein the quaternary ammonium compound is a combination of quaternary ammonium compounds selected from the group consisting of n-alkyl dimethyl benzyl ammonium chloride, di-n-octyl dimethyl ammonium chloride, dodecyl dimethyl ammonium chloride, n-alkyl dimethyl benzyl ammonium saccharinate, and 3-(trimethoxysilyl) propyldimethyloctadecyl ammonium chloride.

8. The topical skin product according to claim 1, wherein the topical skin product is a hand sanitizer, a body treatment, a sun tan product, a hair product, a body moisturizer, a cosmetic face product, or a treatment ointment.

9. The topical skin product according to claim 1, wherein the topical skin product is in a form of a gel, liquid, cream, lotion, aerosol, or roll-on.

10. The topical skin product according to claim 1, wherein the retention ingredient serves as a host for an additive selected from the group consisting of a moisture bead, UV protectant, antimicrobial, skin health additive, and combination thereof.

11. A method of making a topical skin product, the method comprising:
providing a topical skin composition; and
adding a retention ingredient to the topical skin composition, wherein the retention ingredient is a an oxazoline homopolymer or an extended or a modified polymer based on an oxazoline homopolymer.

12. The method according to claim 11, wherein the retention ingredient is present in the topical skin product in an amount in a range of greater than 0% to 10%, based on the weight of the topical skin product.

13. The method according to claim 11, wherein the oxazoline homopolymer has a structure of: 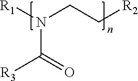
wherein
R₁ is a hydrogen, alkyl, alkenyl, alkoxy, alkylamino, alkynyl, allyl, amino, anilino, aryl, benzyl, carboxyl, carboxyalkyl, carboxyalkenyl, cyano, glycosyl, halo, hydroxyl, oxazolinium mesylate, oxazolinium tosylate, oxazolinium triflate, silyl oxazolinium, phenolic, polyalkoxy, quaternary ammonium, thiol, or thioether group;
R₂ is a hydrogen, alkyl, alkenyl, alkoxy, alkylamino, alkynyl, allyl, amino, anilino, aryl, benzyl, carboxyl, carboxyalkyl, carboxyalkenyl, cyano, glycosyl, halo, hydroxyl, oxazolinium mesylate, oxazolinium tosylate, oxazolinium triflate, silyl oxazolinium, phenolic, polyalkoxy, quaternary ammonium, thiol, or thioether group or a macrocyclic structure;
R₃ is a hydrogen, alkyl, alkenyl, alkoxy, aryl, benzyl, hydroxyalkyl, or perfluoroalkyl group; and
n is in a range of 1 to 1,000,000.

14. The method according to claim 11, wherein the topical skin composition comprises a carrier and a quaternary ammonium compound.

15. The method according to claim 14, wherein the carrier is selected from the group consisting of water, alcohol, and a combination thereof.

16. The method according to claim 14, wherein the quaternary ammonium compound has a structure of: 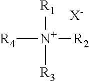
wherein
R¹ is alkyl, alkoxy, or aryl, either with or without heteroatoms, or saturated or non-saturated;
R₂ is alkyl, alkoxy, or aryl, either with or without heteroatoms, or saturated or non-saturated;
R₃ is alkyl, alkoxy, or aryl, either with or without heteroatoms, or saturated or non-saturated;
R₄ is alkyl, alkoxy, or aryl, either with or without heteroatoms, or saturated or non-saturated;
X⁻, an anion, is halogen, sulfonate, sulfate, phosphonate, phosphate, carbonate, bicarbonate, hydroxy, or carboxylate.

17. The method according to claim 14, wherein the quaternary ammonium compound is a combination of quaternary ammonium compounds selected from the group consisting of n-alkyl dimethyl benzyl ammonium chloride, di-n-octyl dimethyl ammonium chloride, dodecyl dimethyl ammonium chloride, n-alkyl dimethyl benzyl ammonium saccharinate, and 3-(trimethoxysilyl) propyl dimethyloctadecyl ammonium chloride.

18. The method according to claim 11, wherein the retention ingredient serves as a host for an additive selected from the group consisting of a moisture bead, UV protectant, antimicrobial, skin health additive, and combination thereof

19. The method according to claim 11, wherein the topical skin product is a hand sanitizer, a body treatment, a sun tan product, a hair product, a body moisturizer, a cosmetic face product, or a treatment ointment.

20. A method of using a topical skin product, the method comprising:
applying to skin the topical skin product comprised of a topical skin composition and a retention ingredient, wherein the retention ingredient is an oxazoline homopolymer or an extended or a modified polymer based on an oxazoline homopolymer.
